(19) United States
(12) Patent Application Publication
(10) Pub. No.: US 2004/0034206 A1
(43) Pub. Date: Feb. 19, 2004

(54) Combination therapy for RNA virus infections involving ribavirin and IMPDH inhibitors

(75) Inventors: Bruce A. Malcolm, Westfield, NJ (US); Gregory R. Reyes, Madison, NJ (US); Sifang Zhou, Germantown, MD (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/449,237

(22) Filed: May 30, 2003

Related U.S. Application Data
(60) Provisional application No. 60/384,658, filed on May 31, 2002.
(60) Provisional application No. 60/405,546, filed on Aug. 22, 2002.

(51) Int. Cl. C07H 21/00; C07H 19/00

(52) U.S. Cl. 536/22.1

(57) ABSTRACT

This invention relates to the treatment of viral infections in animals, particularly mammals. The treatment comprises the administration of a pharmaceutical composition comprising combinations of antiviral compounds useful for treating viral infections. The anti-viral compounds are combined in such a manner as to reduce the detrimental side effects of treatment and to enhance the efficacy. In a particular embodiment, the invention provides a method for treating a viral infection in a mammal comprising administering to a mammal in need of such treatment a therapeutically effective amount of a combination of ribavirin in association with (S)-N-3-[3-(3-methoxy-4-oxazol-5-yl-phenyl)ureido]-benzylcarbamic acid tetrahydrofuran-3-yl-ester and pegylated interferon-alpha-2b.

This application claims the benefit of U.S. Provisional Patent Application No. 60/384,658; filed May 31, 2002 and of U.S. Provisional Patent Application No. 60/405,546; filed Aug. 22, 2002; each of which is herein incorporated by reference in its entirety.

### FIELD OF THE INVENTION

This invention relates to the treatment of RNA virus infections in animals, particularly mammals. The treatment comprises the administration of a pharmaceutical composition comprising combinations of antiviral compounds useful for treating viral infections. The antiviral compounds are combined in such a manner as to reduce the detrimental side effects of treatment and to enhance the efficacy.

### BACKGROUND OF THE INVENTION

During the last several decades, Hepatitis C Virus (HCV) has spread through an unsuspecting population to become one of the most compelling human medical problems faced by the scientific community. Today, HCV is recognized as the causative agent for most cases of non-A and non-B hepatitis, with an estimated human seroprevalence of 1% globally. See Choo, et al., Science, 244:359-362 (1989); Kuo, et al., Science, 244:362-364 (1989); Purcell, FEMS Microbiology Reviews, 14:181-192 (1994). Four million individuals may be infected in the United States alone. See Alter and Mast, Gastroenterol. Clin. North Am., 23:437-455 (1994).

Most infections occur without being diagnosed and subsequently develop into chronic conditions unknown to the patient. Generally, upon first exposure to HCV about 20% of infected individuals develop acute hepatitis that usually appears to resolve spontaneously. In most instances (>80%), however, the virus establishes a chronic infection that persists for decades. This usually results in recurrent and progressively worsening liver inflammation, which often leads to more severe disease states such as cirrhosis and hepatocellular carcinoma. See Kew, FEMS Microbiology Reviews, 14: 211-220 (1994); Saito, et al., Proc. Natl. Acad. Sci. USA 87: 6547-6549 (1990).

As with most viral infections, the treatment options are limited in number and efficacy. Until recently, the only therapy available for treating chronic HCV infection was interferon-alpha (IFN-α) monotherapy. Interferon is used because it can inhibit viral replication in infected cells and also improve liver function in some people. Not all patients are responsive to interferon-alpha treatment, which has limited long term efficacy and a response rate of about 25% depending on the particular genotype of virus.

Interferon therapy is difficult for patients because of the severe side effects. For example, treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. The normal side effects for interferon-alpha are listed in the package insert for INTRON-A interferon alfa-2b, recombinant, published in October of 1994 by Schering Corporation, Kenilworth N.J. Because interferon mono-therapy has limited efficacy and frequent adverse effects, a more effective regimen was needed.

Ribavirin was independently proposed as a monotherapy treatment for chronic hepatitis C infection (Thomas et al. AASLD Abstracts, Hepatology Vol. 20, NO. 4, Pt 2, Number 440, 1994). However, monotherapy treatment was found relatively ineffective and it has undesirable side effects. The normal side effect of ribavirin is hemolytic anemia, which can be severe and debilitating.

Combination therapy of alpha interferon and ribavirin has been proposed and carried out. The results suggest that the combination therapy is more effective than either monotherapy. See Wedemeyer, H., et al. J. Hepatology 29:1010-1014 (1998). Unfortunately, the particular response rate depends on the genotype of virus a person is infected with, but in general only about 40% of patients benefit from this combination therapy and develop a sustained response. Another limitation to combination therapy is that at the current dosages, the undesirable side effects of both interferon and ribavirin are manifested, thus making the treatment difficult for many patients to complete.

The use of pegylated interferon has improved the response rate of HCV patients to the approved interferon and ribavirin combination therapy. This is believed to be through providing more stable effective interferon concentrations during the therapy. Unfortunately, the side effects remain severe.

There is a need for improved methods for treating chronic Hepatitis C Virus infection with a combination of alpha interferon and ribavirin. Improved methods are needed, which provide increased efficacy or, which maintain the current level of efficacy with reduced side effects.

### SUMMARY OF THE INVENTION

The present inventors have discovered that combining ribonuleoside analogs, and inhibitors of enzymes that regulate cellular GTP pools can produce a synergistic effect that allows antiviral therapy at lower doses of those inhibitors and the accompanying side effects, or alternatively, increased efficacy at the same inhibitor doses.

The present inventors have also discovered that combining ribavirin, and IMPDH inhibitors can produce a synergistic effect that allows antiviral therapy at lower doses of ribavirin, or alternatively, increased efficacy at the same ribavirin dose.

Specifically, the present inventors have discovered that using a combination of ribavirin and an inhibitor of the IMPDH enzyme such as (S)-N-3-[3-(3-methoxy-4-oxazol-5-yl-phenyl)ureido]-benzylcarbamic acid tetrahydrofuran-3-yl-ester produces a synergistic anti-viral effect for the treatment of Hepatitis C Virus infections. Moreover, use of this same (S)-N-3-[3-(3-methoxy-4-oxazol-5-yl-phenyl)ureido]-benzylcarbamic acid tetrahydrofuran-3-yl-ester facilitates a reduction in the side toxicity of ribavirin. The methods of this invention may facilitate the use of up to 90% less ribavirin to achieve effective treatment of HCV infection.

Accordingly, the present invention provides a method for treating a viral infection in a mammal comprising administering a therapeutically effective amount of a combination of a ribonucleoside analog in association with an inhibitor of an enzyme that regulates cellular GTP pools. In another preferred embodiment, the ribonucleoside analog is ribavirin or a derivative thereof or a pharmaceutically acceptable salt of the derivative thereof. In an alternate embodiment, the ribonucleoside analog is a mutagen.

The present invention provides a method for treating a viral infection in a mammal comprising administering a therapeutically effective amount of a combination of a ribonucleoside analog in association with an inhibitor of IMPDH. In a certain embodiment, the inhibitor of IMPDH is an uncompetitive inhibitor. In a certain embodiment of the present invention, the inhibitor of IMPDH is mycophenolic acid or a derivative thereof or a pharmaceutically acceptable salt thereof. In a preferred embodiment of the present invention, the inhibitor of IMPDH is (S)-N-3-[3-(3-methoxy-4-oxazol-5-yl-phenyl)ureido]-benzylcarbamic acid tetrahydrofuran-3-yl-ester.

The present invention also provides a method for treating a viral infection in a mammal comprising administering a therapeutically effective amount of a combination of a ribonucleoside analog in association with an inhibitor of IMPDH. In a certain embodiment, the combination further comprises an interferon. In a preferred embodiment of the present invention, the interferon is interferon-alpha. In a more preferred embodiment of the present invention, the interferon is pegylated interferon alpha-2b.

In a preferred embodiment, the susceptible viral infection being treated is Hepatitis C Virus infection. In another embodiment, the susceptible viral infection being treated is a virus selected from the group consisting of West Nile Virus, Dengue Virus, Yellow Fever Virus, Bovine Viral Diarrhea Virus and Venezuelan Equine Encephalitis Virus.

In a preferred embodiment, the present invention provides a method for treating a viral infection in a mammal comprising administering to a mammal in need of such treatment a therapeutically effective amount of a combination of ribavirin in association with (S)-N-3-[3-(3-methoxy-4-oxazol-5-yl-phenyl)ureido]-benzylcarbamic acid tetrahydrofuran-3-yl-ester and pegylated interferon-alpha-2b.

In a preferred embodiment, the therapeutically effective amount of the combination therapy of ribavirin in association with (S)-N-3-[3-(3-methoxy-4-oxazol-5-yl-phenyl)ureido]-benzylcarbamic acid tetrahydrofuran-3-yl-ester and interferon-alpha is administered for a period of about 24 weeks for patients with HCV genotype 2 or 3, or for a period of about 48 weeks for patients with HCV genotype 1.

In a certain embodiment, the present invention provides methods of decreasing the side effects associated with ribavirin antiviral therapy in a mammal comprising administering a therapeutically effective amount of ribavirin in association with an inhibitor of IMPDH. In a preferred embodiment the inhibitor of IMPDH is (S)-N-3-[3-(3-methoxy-4-oxazol-5-yl-phenyl)ureido]-benzylcarbamic acid tetrahydrofuran-3-yl-ester. In a certain embodiment, the combination therapy further comprises an interferon. In a preferred embodiment, the interferon is interferon-alpha. In a further preferred embodiment, the interferon is a pegylated interferon alpha-2b.

In a preferred embodiment, the present invention provides a method for decreasing the side effects of a ribavirin antiviral therapy in a mammal comprising administering to a mammal in need of such decreasing, a therapeutically effective amount of a combination of ribavirin in association with (S)-N-3-[3-(3-methoxy-4-oxazol-5-yl-phenyl)ureido]-benzylcarbamic acid tetrahydrofuran-3-yl-ester and pegylated interferon-alpha.

The present invention provides a method for decreasing the side effects associated with ribavirin antiviral therapy in a mammal comprising administering from about 60 mg/day to about 600 mg/day amount of ribavirin in association with an inhibitor of IMPDH. In a preferred embodiment of the invention, the inhibitor of IMPDH is (S)-N-3-[3-(3-methoxy-4-oxazol-5-yl-phenyl)ureido]-benzylcarbamic acid tetrahydrofuran-3-yl-ester. In another embodiment, the method further comprises administering a therapeutically effective amount of an interferon. In a preferred embodiment, the interferon is interferon-alpha. In a further preferred embodiment, the interferon is a pegylated interferon alpha-2b.

The present invention also provides a method for increasing the efficacy of ribavirin antiviral therapy in a mammal comprising administering from about 600 mg/day to about 1200 mg/day amount of ribavirin in association with an inhibitor of IMPDH. In a preferred embodiment of the invention, the inhibitor of IMPDH is (S)-N-3-[3-(3-methoxy-4-oxazol-5-yl-phenyl)ureido]-benzylcarbamic acid tetrahydrofuran-3-yl-ester. In another embodiment, the method further comprises administering a therapeutically effective amount of an interferon. In a preferred embodiment, the interferon is interferon-alpha. In a further preferred embodiment, the interferon is a pegylated interferon alpha-2b.

The present invention further provides a method for decreasing the side effects of a ribavirin antiviral therapy in a mammal comprising administering to a mammal in need of such decreasing a dose of from about 60 mg/day to about 600 mg/day of ribavirin in association with (S)-N-3-[3-(3-methoxy-4-oxazol-5-yl-phenyl)ureido]-benzylcarbamic acid tetrahydrofuran-3-yl-ester and pegylated interferon-alpha-2b.

The present invention also provides a pharmaceutical composition comprising: (a) a therapeutically effective amount of ribavirin; and (b) a therapeutically effective amount of (S)-N-3-[3-(3-methoxy-4-oxazol-5-yl-phenyl)ureido]-benzylcarbamic acid tetrahydrofuran-3-yl-ester. In a preferred embodiment, the composition further comprises a pharmaceutically acceptable carrier. In a further embodiment, the carrier is in a solid form. In a preferred embodiment, the compostion comprises from about 400 mg to about 800 mg of ribavirin or a derivative thereof or a pharmaceutically acceptable salt therof.

In a certain embodiment of the present invention, the amount of ribavirin ranges from about 1 mg to about 6000 mg in the pharmaceutical composition, preferably the amount of ribavirin ranges from about 100 mg to about 1200 mg in the pharmaceutical composition, more preferably the amount of ribavirin ranges from about 100 mg to about 600 mg in the pharmaceutical composition.

In another embodiment, the present invention provides a pharmaceutical composition comprising a pharmaceutically acceptable carrier and from about 1 mg to about 6000 mg of the uncompetitive inhibitor of IMPDH, (S)-N-3-[3-(3-methoxy-4-oxazol-5-yl-phenyl)ureido]-benzylcarbamic acid tetrahydrofuran-3-yl-ester. In a preferred embodiment, the amount of (S)-N-3-[3-(3-methoxy-4-oxazol-5-yl-phenyl)ureido]-benzylcarbamic acid tetrahydrofuran-3-yl-ester is in the range of from about 150 mg to about 5000 mg. In a certain embodiment, the pharmaceutical composition further contains an interferon, preferably pegylated interferon-alpha, more preferably pegylated interferon-alpha-2b.

In one embodiment of the invention, the pharmaceutical composition also comprises a pharmaceutically acceptable carrier in a solid form and the carrier is selected from the group consisting of lactose, sucrose, gelatin and agar.

### DETAILED DESCRIPTION

In a preferred embodiment, the methods of the present invention are used for treating infections caused by RNA viruses. The methods of the present invention are especially suited for treating infections arising from RNA viruses having a positive stranded genome and the various associated types. The phrase “positive stranded genome” of a virus is one in which the genome, whether RNA or DNA, is single-stranded and which encodes a viral polypeptide(s). Examples of positive stranded RNA viruses include the virus families Flaviviridae, Togaviridae, Coronaviridae, Retroviridae, Picornaviridae, and Caliciviridae. See Fields & Knipe (1986) “Fundamental Virology” (Raven Press, NY). Examples of virus families and specific members of the family, where the methods of present invention are useful are shown below:

Flaviviridae such as Hepatitis C Virus, West Nile Virus, Dengue Virus, Yellow Fever Virus, Japanese encephalitis virus, St. Louis encephalitis virus, central European encephalitis virus and Bovine Viral Diarrhea Virus. See Fields Virology 3^{rd} edition, Edited by B. N. Fields, D. M. Knipe, and P. M. Howley, Lippencott Raven, Philadelphia Pa. (1996), Chapters 30-33 for Flaviviridae (pp. 931-1074), the disclosure of which is hereby incorporated by reference in its entirety.

Togaviridae such as Rubella, Western equine encephalitis virus, Venezuelan Equine Encephalitis Virus, Eastern equine encephalitis virus, Semiliki Forest virus, and Sinbis virus. See Fields Virology 3^{rd} edition, Edited by B. N. Fields, D. M. Knipe, and P. M. Howley, Lippencott Raven, Philadelphia Pa. (1996), Chapters 27-29 for Togaviridae (pp. 825-930), the disclosure of which is hereby incorporated by reference in its entirety.

Coronaviridae, such as human respiratory coronaviruses HCV-229E & OC43. See Fields Virology 3^{rd} edition, Edited by B. N. Fields, D. M. Knipe, and P. M. Howley, Lippencott Raven, Philadelphia Pa. (1996), Chapters 34-35 for Coronaviridae (pp. 1075-1104), the disclosure of which is hereby incorporated by reference in its entirety.

Retroviridae such as HIV, and HTLV-I & II. See Fields Virology 3^{rd} edition, Edited by B. N. Fields, D. M. Knipe, and P. M. Howley, Lippencott Raven, Philadelphia Pa. (1996), Chapters 58-62 for Retroviridae (pp. 1767-1996), the disclosure of which is hereby incorporated by reference in its entirety.

Picornaviridae such as human rhinoviruses, poliovirus, coxsackievirus A & B, hepatitis A virus, Echovirus, encephalomyocarditis virus and Theiler's virus. See Fields Virology 3^{rd} edition, Edited by B. N. Fields, D. M. Knipe, and P. M. Howley, Lippencott Raven, Philadelphia Pa. (1996), the disclosure of which is hereby incorporated by reference in its entirety.

Caliciviridae such as the Norwalk Group of viruses. See Fields Virology 3^{rd} edition, Edited by B. N. Fields, D. M. Knipe, and P. M. Howley, Lippencott Raven, Philadelphia Pa. (1996), Chapter 25 for Calciviridae (pp. 784-804), the disclosure of which is hereby incorporated by reference in its entirety.

The term “RNA virus infection” as used herein refers to a patient who is infected with an RNA virus. A salient characteristic of viruses with RNA genomes is that they have relatively high rates of spontaneous mutation reportedly on the order of 10⁻³ to 10⁻⁴ per incorporated nucleotide. See Fields & Knipe (1986) “Fundamental Virology” (Raven Press, NY). Since heterogeneity and fluidity of genotype are inherent in RNA viruses, there are multiple types/subtypes, within the species which may be virulent or avirulent. For example, the propagation, identification, detection, and isolation of various HCV types or isolates is documented in the literature. The number of the HCV-1 genome and amino acid residues sequences is as described in Choo et al. (1990) Brit. Med. Bull., 46:423-441.

Hepatitis C Virus

Hepatitis C Virus (HCV) infection is of particular concern to public health worldwide. In the United States alone, an estimated four million individuals are chronically infected with HCV. HCV, the major etiologic agent of non-A, non-B hepatitis, is transmitted primarily by transfusion of infected blood and blood products. Prior to the introduction of anti-HCV screening in mid-1990, HCV accounted for 80-90% of post-transfusion hepatitis cases in the United States. A high rate of HCV infection is also seen in individuals with bleeding disorders or chronic renal failure, groups that have frequent exposure to blood and blood products.

Acute infection with HCV results in persistent viral replication and progression to chronic hepatitis in approximately 90% of cases. For many patients, chronic HCV infection results in progressive liver damage and the development of cirrhosis. In patients with an aggressive infection, cirrhosis can develop in as little as two years, although a time span of 10-20 years is more typical. In 30-50% of chronic HCV patients, liver damage may progress to the development of hepatocellular carcinoma. In general, hepatocellular carcinoma is a late occurrence and may take greater than 30 years to develop. See Bisceglie et al., Semin. Liver Dis. 15:64-69 (1995). The relative contribution of viral or host factors in determining disease progression is not clear.

Hepatitis C Virus is an enveloped, positive stranded, RNA virus from the Flaviviridae family. The HCV genome of approximately 9.5 kb encodes a polyprotein of approximately 3000 amino acids. See Choo, et al. Proc. Natl. Acad. Sci. USA, 88:2451-2455 (1991); Kato, et al., Proc. Natl. Acad. Sci. USA, 87:9524-9528 (1990); Takamizawa, et al., J. Virol., 65:1105113 (1991). The viral genome further consists of a lengthy 5′ untranslated region (UTR), and a short 3′ UTR. The 5′ UTR is the most highly conserved part of the HCV genome and is important for the initiation and control of polyprotein translation. Translation of the HCV genome is initiated by a cap-independent mechanism known as internal ribosome entry. This mechanism involves the binding of ribosomes to an RNA sequence known as the internal ribosome entry site (RES). An RNA pseudoknot structure has recently been determined to be an essential structural element of the HCV IRES. The polyprotein precursor is cleaved by both host and viral proteases to yield mature viral structural and nonstructural proteins.

The HCV non-structural (NS) proteins provide catalytic machinery for viral replication and are derived by proteolytic cleavage of the polyprotein. See Bartenschlager, et al., J. Virol., 67:3835-3844 (1993); Grakoui, et al., J. Virol, 67:2832-2843 (1993); Grakoui, et al., J. Virol., 67:1385-1395 (1993); Tomei, et al., J. Virol., 67:4017-4026 (1993). Viral structural proteins include a nucleocapsid core protein (C) and two envelope glycoproteins, E1 and E2. HCV also encodes two proteinases, a zinc-dependent metalloproteinase, encoded by the NS2-NS3 region, and a serine proteinase encoded in the NS3 region. These proteinases are required for cleavage of specific regions of the precursor polyprotein into mature peptides. The carboxyl half of nonstructural protein 5, NS5B, contains the RNA-dependent RNA polymerase.

HCV isolates exhibit marked sequence diversity and have been grouped according to phylogenetic analysis into six different genotypes, which together form the genus Hepacivirus within the family Flaviviridae. There is a correlation between HCV genotype and response to interferon therapy. See Enomoto et al., N. Engl. J. Med. 334:77-81 (1996); Enomoto et al., J. Clin. Invest. 96:224-230 (1995). For example, the response rate in patients infected with HCV-1b is less than 40%. Similar low response rates for patients infected with prototype United States genotype, HCV-1a, have also been reported. See Lino et al, Intervirology 37:87-100 (1994). In contrast, the response rate of patients infected with HCV genotype-2 is nearly 80%. See Fried et al., Semin. Liver Dis. 15:82-91 (1995).

Hepatitis C Virus frequently results in a chronic infection that progressively impairs liver function. For example, a person suffering from chronic hepatitis C infection may exhibit one or more of the following signs or symptoms: (a) elevated serum alanine aminotransferase (ALT), (b) positive test for anti-HCV antibodies, (c) presence of HCV as demonstrated by a positive test for HCV-RNA, (d) clinical stigmata of chronic liver disease, (e) hepatocellular damage. Such criteria may not only be used to diagnose hepatitis C, but can be used to evaluate a patient's response to drug treatment.

Interferon therapy can help normalize liver function through its antiviral activity. Elevated ALT and aspartate aminotransferase (AST) are known to occur in uncontrolled hepatitis C, and a complete response to treatment is generally defined as the normalization of these serum enzymes, particularly ALT. See Davis et al., New Eng. J. Med. 321:1501-1506 (1989). ALT is an enzyme released when liver cells are destroyed and is symptomatic of HCV infection. Interferon induces synthesis of the enzyme 2′,5′-oligoadenylate synthetase (2′5′OAS), which in turn, causes the degradation of the viral mRNA. Increases in serum levels of the 2′5′OAS coincide with decreases in ALT levels.

Histological examination of liver biopsy samples may be used as a second criteria for evaluation. See, e.g., Knodell et al., Hepatology 1:431-435 (1981), whose Histological Activity Index (portal inflammation, piecemeal or bridging necrosis, lobular injury and fibrosis) provides a scoring method for disease activity.

The term “patients having hepatitis C infections” as used herein means any patient-including a pediatric patient-having hepatitis C and includes treatment-naive patients having hepatitis C infections and treatment-experienced patients having hepatitis C infections as well as those pediatric, treatment-naive and treatment-experienced patients having chronic hepatitis C infections.

These patients having chronic hepatitis C include those who are infected with multiple HCV genotypes including type 1 as well as those infected with HCV genotype 2 and/or 3. Hepatitis C Virus infections may be detected by measuring the patient's anti-HCV antibody response and the diagnosis is usually verified using polymerase chain reaction amplification of viral RNA.

The term “treatment-naive patients having hepatitis C infections” as used herein means patients with hepatitis C who have never been treated with ribavirin or any interferon, including but not limited to interferon-alpha, or pegylated interferon alpha.

The term “treatment-experienced patients having hepatitis C infections” as used herein means patients with hepatitis C who have been treated with ribavirin or any interferon, including but not limited to interferon-alpha, or pegylated interferon alpha, including relapsers and non-responders.

The term “patients having chronic hepatitis C infections” as used herein means any patient having chronic hepatitis C and includes “treatment-naive patients” and “treatment-experienced patients” having chronic hepatitis C infections, including but not limited to relapsers and non-responders.

The term “relapsers” as used herein means treatment-experienced patients with hepatitis C who have relapsed after initial response to previous treatment with interferon alone, or in combination with ribavirin.

The term “non-responders” as used herein means treatment-experienced patients with hepatitis C who have not responded to prior treatment with any interferon alone, or in combination with ribavirin.

In a preferred embodiment, the present invention provides an improved method for treating patients having hepatitis C infection by administering a therapeutically effective amount of a combination therapy to ameliorate the ribavirin-related hemolysis so as to allow such patients to continue using a therapeutically effective amount of combination therapy until a sustained virologic response is achieved. The combination therapy includes, but is not limited to (a) a therapeutically effective amount of (S)-N-3-[3-(3-methoxy-4-oxazol-5-yl-phenyl)ureido]-benzylcarbamic acid tetrahydrofuran-3-yl-ester, ribavirin and interferon alpha-2b or (b) a therapeutically effective amount of (S)-N-3-[3-(3-methoxy-4-oxazol-5-yl-phenyl)ureido]-benzylcarbamic acid tetrahydrofuran-3-yl-ester, ribavirin and a pegylated interferon alpha-2b such as approved by the FDA as well as other (S)-N-3-[3-(3-methoxy-4-oxazol-5-yl-phenyl)ureido]-benzylcarbamic acid tetrahydrofuran-3-yl-ester, ribavirin and interferon alpha combination therapies under clinical development or those described hereinafter.

The combination therapy is continued for a time period of at least about 24 weeks, more preferably is at least about 48 weeks or 72 weeks, and most preferably is at least about 48 weeks.

The present method for treating patients having chronic hepatitis C infections allows delivery of therapeutically effective amount of the combination of ribavirin, an IMPDH inhibitor and interferon-alpha, sufficient to substantially lower detectable HCV-RNA serum levels, preferably by at least two powers of ten, i.e., at least 10² lower than the initial HCV-RNA serum level, and more preferably eradicate detectable HCV-RNA serum levels.

The term “susceptible viral infections” as used herein means viral infections caused by a wide range of RNA viruses, including, but not limited to, the families of viruses such as Flaviviridae, Togaviridae, Coronaviridae, Retroviridae, Picornaviridae, and Caliciviridae families, which includes Hepatitis C Virus, Dengue Virus, West Nile Virus, Yellow Fever Virus, Venezuelan Equine Encephalitis Virus and St. Louis encephalitis virus.

Typical suitable susceptible viral infections include Hepatitis C Virus, West Nile Virus, Dengue Virus, Yellow Fever Virus, Venezuelan encephalitis virus, St. Louis encephalitis virus, influenza A and B viral infections; parainfluenza viral infections, respiratory syncytial virus (“RSV”) infections such as RSV bronchiolitis and RSV pneumonia especially such RSV infections in children and infants as well as RSV pneumonia in patients with preexisting cardiopulmonary disease, measles viral infections, Lassa fever viral infections, Korean Haemorrhagic fever infections, hepatitis B viral (HBV) infections, Crimean•Congo-Haemorrhagic and HIV-1 infections, encephalitis infections such as caused by Kunjin virus or the St. Louis encephalitis infections as well as viral infections found in immunocompromised patients.

Other susceptible viral infections are those which respond to ribavirin. Further examples of susceptible viral infections are disclosed in U.S. Pat. No. 4,211,771 at column 2, line 21 to column 3 line 37; doses and dose regimens and formulations are disclosed at column 3, line 4 to column 9, line 5; see also Canadian Patent No. 1,261,265. Sidwell, R. W., et al. Pharmacol. Ther., 1979, Vol 6 pp. 123-146 discloses that the in vivo antiviral experiments conducted with ribavirin generally confirm broad-spectrum antiviral activity seen in vitro and states that the efficacy of ribavirin is quite dependent upon the site of infection; the manner of treatment; the age of the animal and the virus dosage utilized. Tables 4 and 5 on page 127 list the RNA and DNA virus infections significantly inhibited in vivo by ribavirin. See Sidwell, R. W., et al. Pharmacol. Ther., 1979, Vol 6 pp. 123-146.

West Nile Virus

West Nile Virus (WNV) is a member of the Flavivirus genus of the Flaviviridae family. WNV is antigenically related to the Saint Louis Encephalitis virus (SLE), Japanese encephalitis virus (JE) and Kunjin virus. WNV is believed to have entered the United States in 1999 and has since caused significant human and veterinary disease and death. See J. F. Anderson et al., Science, 286:2331-2333 (1999) and RS. Lanciotti et al., Science, 286:2333-2337 (1999).

The flavivirus genome of which WNV is a representative member is a single plus-strand RNA of approximately 11 kb in length that encodes 10 viral proteins in a single open reading frame. The encoded polyprotein is translated and co- and post-translationally processed by viral and cellular proteases into three structural proteins (the capsid protein C; the membrane protein M, which is formed by furin-mediated cleavage of prM; and the envelope protein E) and seven nonstructural proteins (the glycoprotein NS1, NS2a, the protease cofactor NS2b, the protease and helicase NS3, NS4a, NS4b, and the polymerase NS5). See Chambers, T. J., C. et al., Annu. Rev. Microbiol. 44:649-688 (1990). The 5′ and 3′ untranslated regions (UTRs) of the genomic RNA are approximately 100 and 400 to 700 nucleotides (nt) in length, respectively, and the terminal nucleotides of both the 5′ and the 3′ UTRs can form highly conserved secondary and tertiary structures.

WNV grows in numerous primary and continuous cell lines. For example, WNV has been grown in chick and duck embryo primary cells and various human, primate (vero cells), rodent (BHK-21 cells), swine and insect continuous cell lines (Aedes albopictus C6/36 cells).

A full length infectious molecular clone of the WNV Lineage I, which is responsible for the recent epidemic in the United States, was recently reported. See P. Y. Shi et al., J. Virology, 76:5847-5856 (2002), the disclosure of which is hereby incorporated by reference in its entirety. The WNV clone provides the basis for a reverse genetic system because it allows us to make mutations at the level of DNA, then generate infectious RNA from the mutated DNA, transfect the infectious RNA back into cells and then to correlate sequence with pathogenicity, inhibition of infection or any other observable characteristic. Such a system can be useful in developing methods of treatment for WNV. See P. Y. Shi et al., J. Virology, 76:5847-5856 (2002).

The full length cDNA clone of approximately 11,029 base pairs of WNV is constructed by assembling four fragments generated using RT-PCR from the RNA genome. The WNV cDNA is cloned into plasmid pBR322 and positioned under the control of the T7 promoter and can be amplified by growth in Eschericha coli HB101. Cells may be infected with the cloned virus by first transcribing the DNA clone to generate viral genomic RNA and then transfecting the genomic RNA into BHK-21 cells. See P. Y. Shi et al., J. Virology, 76:5847-5856 (2002). Progeny virus may then be produced from cells transfected with the genomic RNA corresponding to the WNV cDNA clone. Titers of progeny virus were reported in the range of 1×10⁹ to 5×10⁹ plaque forming units per ml (PFU/ml). See P. Y. Shi et al., J. Virology, 76:5847-5856 (2002).

Dengue Fever Virus

Dengue Virus (DV) is an increasingly important member of the Flaviviridae family and is a public health threat world-wide. Dr. Benjamin Rush, the physician friend of President John Adams described what seems like a Dengue Virus like epidemic in the United States in Philadelphia in 1780. It is now known that there are four serologic types of Dengue Virus (types 1-4), which form a distinct antigenic complex within the Flaviviridae family. All four Dengue Virus types cause epidemics. More rarely there are epidemics of very severe dengue hemorrhagic fever/dengue shock syndrome (DHF/DSS). The four types can be distinguished on the basis of plaque reduction neutralization tests and reactivity to specific monoclonal antibodies. Nucleotide sequence analysis reveals that Dengue Types 1 through 4 are approximately 63%-68% homologous overall at the amino acid level. In contrast, the Dengue Viruses are between 44%-51% homologous to the yellow fever and West Nile Viruses overall at the amino acid level.

Dengue Viruses can be grown in many different primary and continuous cell cultures. See Fields Virology 3^{rd} edition, Edited by B. N. Fields, D. M. Knipe, and P. M. Howley, Lippencott Raven, Philadelphia Pa. (1996), Chapters 30-33 for Flaviviridae (pp. 931-1074), the disclosure of which is hereby incorporated by reference in its entirety. For example, DV has been grown in human cells (BSC-1, HL-CZ), primate cells (LLC-MK2, Vero, primary monkey kidney), hamster cells such as BHK-21 and mosquito cells (C6/36). Usually, the virus must be adapted and subjected to multiple passages to achieve high titers and to demonstrate cytopathic effects in culture. Dengue Viruses may be grown in the brains of suckling mice and hamsters following intracerebral inoculation. In addition, DV may be grown to high titers in certain mosquitoes by intrathroracic or intracerebral inoculation. Examples of mosquito hosts useful for growing DV include Aedes spp. and Toxorhynchites spp.

In humans, DV fever generally results in a self-limiting infection. The initial clinical symptoms arise after a 2-7 day incubation period and include a high fever, headache, retrobulbar pain, lumbosacral pain, conjunctival congestion and facial flushing. Subsequently, the patient develops bone pain or generalized myalgia, nausea, vomiting, weakness, prostration and anorexia. The disease course may be accompanied by a rash that spreads over the trunk, face and limbs. The final phase of the fever may include a lymphadenopathy, granulocytopenia and platelets below 100 K/mm³. Dengue Virus infection may lead to hemorrhagic disease or be associated with numerous neurologic manifestations.

Yellow Fever Virus

Historically, Yellow Fever (YF) virus is important because devastating YF epidemics have been recorded in Europe and in the Americas since the 17^{th} century. Due to the prominence of YF virus throughout modern history it has served as a model virus for studying the Flavivirus genus. See Fields Virology 3^{rd} edition, Edited by B. N. Fields, D. M. Knipe, and P. M. Howley, Lippencott Raven, Philadelphia Pa. (1996), Chapters 30-33 for Flaviviridae (pp. 931-1074), the disclosure of which is hereby incorporated by reference in its entirety. Much of what is known regarding genome structure and Flavivirus replication has been elucidated using YF virus. The severity of the impact of YF virus on humans is more limited today because there is an effective and relatively safe live vaccine available. The vaccine is based on the first isolated African YF virus strain, known as 17D.

Yellow Fever Virus is not closely related antigenically to other Flaviviruses as measured by neutralization assays and monoclonal antibody reactivity. RNA fingerprinting and nucleotide sequencing indicates that YF virus may be distinguished into at least three separate geographic topotypes. See Fields Virology 3^{rd} edition, Edited by B. N. Fields, D. M. Knipe, and P. M. Howley, Lippencott Raven, Philadelphia Pa. (1996), Chapters 30-33 for Flaviviridae (pp. 931-1074), the disclosure of which is hereby incorporated by reference in its entirety. Further, the geographic topotypes may be subdivided into genotypes according to nucleotide sequence of the E gene.

Yellow Fever Virus can be grown in many primary and continuous cell cultures. For example, YF virus may be propagated in monkey kidney continuous cell cultures such as vero, MA-104 and LLC-MK2 cells. In addition, baby hamster kidney (BHK) and rabbit kidney (MA-111) cells support YF virus growth. The vaccine strain 17D may be grown in human cell lines such as HeLa, Chang liver cells, and KB cells. Finally, YF virus can be isolated in mosquito cell cultures or by intrathroracic inoculation of Aedes aegypti or subpassaged into infant mice.

The clinical course of YF virus in humans is similar to other Flaviviruses. An incubation period of 3 to 6 days is followed by development of fever, chills, headache, lumbosacral pain, myalgia, anorexia, nausea, vomiting and gingival hemorrhages. Next, there is usually a short remission for about 24 hours. Remission is followed by a recurrence of symptoms including increased vomiting, epigastric pain, jaundice and prostration. Death may occur in 20-50% of severe cases and is usually preceeded by deepening jaundice, hemorrhages, rising pulse, hypotension, oliguria and azotemia. Terminal signs include hypothermia, agitated delirium, intractable hiccups, stupor and coma.

Venezuelan Equine Encephalitis Virus

Venezuelan Equine Encephalitis Virus (VEEV) is a member of the Alphavirus genus of the Togaviridae virus family. See Fields Virology 3^{rd} edition, Edited by B. N. Fields, D. M. Knipe, and P. M. Howley, Lippencott Raven, Philadelphia Pa. (1996), Chapters 27-29 for Togaviridae (pp. 825-930). Venezuelan Equine Encephalitis Virus is an equine virus that can infect man and may produce a fatal encephalitis in infected persons. It was first isolated in Venezuela during an epizootic in the 1930's. There are specific strains of VEE that cause epizootics and these have been shown to replicate to high titers in equines and can infect man. For example, VEE strains IA, IB and IC have been isolated during horse epizootics and can be transmitted by multiple mosquito species to man. In contrast, VEE strains ID, IE and IF are associated with enzootic activity, do not replicate to high titer in horses and appear to be transmitted selectively by mosquitos of the Culex genus.

The genome and virion of Venezuelan Equine Encephalitis Virus is not completely characterized. However, VEE is an enveloped RNA virus with viral genome of between 11 and 12 kbp. The 5′ end of the of the VEEV genome is modified with a methylated cap and the 3′ end contains a variable length poly(A) tract. In the virion, a single capsid protein “C” is associated with the RNA genome. The capsid is surrounded by a lipid envelope containing an array of protein spikes composed of heterodimers of two transmembrane glycoproteins E1 and E2.

The clinical path for VEE infections involve first a 1 to 6 day incubation period followed by a rapid onset of high fever and malaise. Most cases present with a fever ranging from 102° F. to 105° F., chills, myalgia, headache, and lethargy. Many cases express photophobia, hyperesthesia, vomiting and prostration. Acute symptoms persist for 2-5 days. More than 50% of those infected acquire some form of disease.

Venezuelan Equine Encephalitis Virus can be grown in many primary and continuous cell cultures. For example, VEE virus may be propagated in monkey kidney continuous cell cultures such as vero, and chick embryo fibroblasts (CEF) cells. In addition, baby hamster kidney (BHK) support VEE virus growth.

Bovine Viral Diarrhea Virus

Acute infection of immunocompetent cattle with Bovine Viral Diarrhea Virus (BVDV) can result in a wide range of clinical syndromes. Although not entirely clear, the outcome of an acute infection is probably related to several factors including strain of virus, age of host, immune and physiologic status of the host, and the presence of other pathologic agents.

The majority of acute BVDV infections are caused by noncytopathic viruses. Cattle acutely or persistently infected with BVDV are the primary source of virus. Infected animals shed virus in nasal and oral secretions, feces and urine. The primary virus entrance route is probably oral or nasal Other less important routes of entry may include infected semen, biting insects, and contaminated instruments. Following entry and contact with the mucosal lining of the mouth or nose, initial replication occurs in epithelial cells with a predilection for the palatine tonsils. Isolation of virus from serum or leucocytes is generally possible between 3 and 10 days post infection. During systemic spread, the virus is able to gain entry to most tissues with a preference for lymphoid tissues.

Following acute infection, it is generally accepted that most clinical outcomes are mild. Mild fevers, diarrhea, and leukopenia have most commonly been described following acute infection. However, some viral strains have been associated with much more severe disease including fatal hemorrhagic diarrhea and fatal thrombocytopenia.

The genetic and antigenic diversity between different strains of BVDV is quite varied. However, it has become clear that two major groups of virus exist which differ significantly with respect to both their genetic and antigenic makeup. These two groups are referred to type I BVDV and type II BVDV. Although differences exist at the molecular level, the major aspects of the pathogenesis of disease caused by type I and type II BVDV appear to be the same. The major difference is that acute infections with type II BVDV have been associated with more severe clinical syndromes. The reasons for this increase in severity are unknown.

Bovine Viral Diarrhea Virus is a member of the genus Pestivirus within the flaviviridae family of virus. The genome of BVDV consists of a single strand of positive sense RNA which is about 12,300 nucleotides long. A single open reading frame exists which is approximately 3900 codons long. The open reading frame is flanked by 5′ and 3′ untranslated regions which are important for the initiation of translation and RNA stability. The open reading frame is translated into a single polyprotein which is then processed by both viral and cellular enzymes into mature viral proteins.

The BVDV genome encodes for both structural and nonstructural proteins. The structural proteins include the capsid protein C (p14) and three glycoproteins Erns, E1, and E2 (gp48, gp25, and gp53). Six nonstructural proteins are encoded by the noncytopathic BVDV genome. Npro (p20) is the first protein produced from the open reading frame. It has papain-like protease activities. The next nonstructural protein produced is NS23 (p125). This protein has several unique characteristics which suggest its involvement in multiple functions. These characteristics include a very hydrophobic domain, a zinc-finger, a protease, and a helicase. Cytopathic BVDV strains have changes within the coding region for p125 which result in the production of the protein NS3 (p80). This protein is unique to the cytopathic BVDV biotype. The NS3 protein of cytopathic BVDV contains the protease and helicase activity of the NS23 protein. Other nonstructural proteins include NS4A (p10), NS4B (p32), and NS5A (p58). Knowledge of these proteins is limited. The final protein produced is NS5B (p75), which is the RNA-dependent-RNA polymerase needed to replicate the viral genome.

Inhibitors of Enzymes that Regulate Ribonucleoside Pools

Many viruses rely on host enzymes for supplying sufficient building blocks such as nucleotides and amino acids to support virus replication. The enzymes that are directly involved in producing nucleotides and also enzymes that regulate the cellular levels of nucleotides (pools), such as GMP and AMP, are especially attractive targets for developing anti-viral therapies. Examples of enzymes that control ribonucleotide pools include adenylosuccinate synthase (Enzyme Commission Number 6.3.4.4); adenylosuccinate lyase EC 4.3.2.2; IMP dehydrogenase EC 1.1.1.205; GMP synthase EC6.3.5.2; 5-phosphoribosyl-1-pyrophosphate synthetase; and glutamine PRPP amidotransferase.

The enzyme IMP dehydrogenase (IMPDH) is of particular interest because it is a critical enzyme in the de novo biosynthetic pathway of purine nucleotides. The Enzyme Commission Number for IMPDH is 1.1.1.205 (EC 1.1.1.205). IMPDH is an excellent target for anti-viral compounds because it is positioned at the branch point for the synthesis of both guanine and adenine nucleotides. As such, IMPDH represents the rate-limiting step in providing de novo supplies of guanine nucleotides, which are essential precursors for both RNA and DNA. In addition to DNA replication and protein synthesis, guanine nucleotides function in many important cellular activities such as signal transduction, regulation of metabolic processes and glycosylation. Perhaps due to the importance of purine nucleotides in so many cellular processes, IMPDH appears to be produced by all living organisms.

It is known that inhibitors of IMPDH activity are useful as antiviral agents. IMPDH catalyzes the NAD⁺-dependent oxidation of inosine monophosphate (IMP) to xanthine monophosphate (XMP), which is subsequently converted to GTP. See R. C. Jackson et al., Nature, 256, pp. 331-333 (1975).

Inhibitors of IMPDH and methods of making them are known in the art. See for example U.S. Pat. No. 5,807,876 to Amistead et al., and U.S. Pat. No. 6,153,398 to Collart et al., the disclosures of which are hereby incorporated by reference in their entirety. The protein crystal structure of IMPDH alone and IMPDH complexed with substrate and inhibitor have been solved and are available for use in designing new inhibitors. See U.S. Pat. No. 6,128,582 to Wilson et al., the disclosure of which is hereby incorporated by reference in its entirety.

One such IMPDH inhibitor is the compound (S)-N-3-[3-(3-methoxy-4-oxazol-5-yl-phenyl)ureido]-benzylcarbamic acid tetrahydrofuran-3-yl-ester. The structure and properties are described in J. Jain et al., Pharmaceutical Sciences 90:625-637 (2000) and W. Markland et al., Antimicrobial Agents and Chemotherapy, 44: 859-866 (2000). This compound is referred to as IMPDH-I throughout this application.

One class of inhibitors are represented by Mycophenolic acid and derivatives which are inhibitors of IMPDH. Mycophenolic acid is commercially available from the Ajinomoto Company of Tokyo, Japan. Mycophenolic acid can be synthesized as described in U.S. Pat. No. 4,452,891, to T. Kida et al., (1984) the disclosure of which is hereby incorporated by reference in its entirety. Many derivatives of mycophenolic acid are known and can be synthesized as described in U.S. Pat. No. 5,380,879 to Sjogren (1995) and U.S. Pat. No. 5,444,072 to Patterson et al., (1995), the disclosures of which are hereby incorporated by reference in their entirety.

Another IMPDH inhibitor which may be used in the present invention is mizoribine (N′-[β-D-ribofuranosyl]-5-hydroxyimidazole-4-carboxamide;

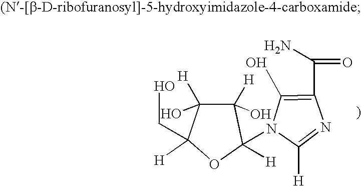

The present invention includes methods for treating hepatitis C and other viral infections (e.g., West Nile Virus, Dengue Virus, Yellow Fever Virus, Bovine Viral Diarrhea Virus and Venezuelan Equine Encephalitis Virus) in a mammal comprising administering mizoribine in combination with a ribonucleoside analogue, such as ribavirin, and/or interferon. Methods for decreasing side-effects associated with ribavirin in a mammal comprising administering mizoribine in combination with ribavirin, and/or interferon are also part of the present invention. Compositions comprising mizoribine and a ribonucleoside analogue (e.g., ribavirin) and, optionally, interferon are also part of the present invention.

Ribavirin

Ribavirin (1-beta-D-Ribofuranosyl-1H-1,2,4-triazole-3-carboxamide) is a broad spectrum antiviral agent having activity against many RNA and DNA viruses. See E. DeClercq, Antiviral agents: Characteristic activity spectrum depending on the molecular target with which they interact, p. 1-55. Academic Press, Inc. New York, N.Y. The free base ribavirin is a nucleoside analog, which undergoes intracellular phosphorylation to the 5′ nucleoside to become a competitive inhibitor of IMPDH. See Entry No. 8199, The Merck Index, Eleventh Edition, Merck & Co. Inc., Rahway, N.J. 1989. The Chemical Abstracts number is CAS-36791-04-5. Ribavirin is sold by ICN pharmaceuticals of Canada under the Brand Name VIRAZOLE™.

The antiviral activity of ribavirin is not derived solely from competitive inhibition of IMPDH. There are other antiviral modes of action of ribarivin suggested in the literature, which may contribute to ribavirin's activity. For example, ribavirin is reported to be effective against Hepatitis C Virus by the following mechanisms: First, the error catastrophe theory holds that misincorporation of ribavirin into the HCV genome results in lethal mutagenesis. See Crotty S, et al., Nature Med. 6:1375-9 (2000); Crotty S., et al. Proc Natl Acad Sci USA. 98:6895-900 (2001); and Maag A, et al., J. Biol. Chem. 276 (49):46094-8 (2001). Second, ribavirin may slow viral replication by directly inhibiting the viral polymerases. See Eriksson B, et al., Antimicrob Agents Chemother 11:946-51 (1977). Third, ribavirin inhibits HCV replication through direct inhibition of the cellular IMPDH enzyme as a competitive inhibitor, thus leading to reduced GTP pools. See Streeter DG, et al., Proc Natl Acad Sci USA 70: 1174-78 (1973). And fourth, ribavirin inhibits HCV by shifting the cytokine expression profile of helper T-cells from a Th 2 profile toward a Th 1 profile. See Tam R C, et al., J Hepatol. 30: 376-82 (1999).

The in vitro inhibitory concentrations of ribavirin are disclosed in Goodman & Gilman's “The Pharmacological Basis of Therapeutics”, Ninth Edition, (1996) McGraw Hill, NY, at pages 1214-1215. Ribavirin is sold under the trade name VIRAZOLE™. Ribavirin dosage and dosage regimens are also disclosed by Sidwell, R. W., et al., Pharmacol. Ther 1979 Vol 6. pp. 123-146 in section 2.2 pp. 126-130. Fernandes, H., et al., Eur. J. Epidemiol., 1986, Vol. 2(1) pp. 1-14 disclose at pages 4-9 dosage and dosage regimens for oral, parenteral and aerosol administration of ribavirin in various preclinical and clinical studies.

In prior treatment of chronic hepatitis C infection with ribavirin monotherapy the usual dosage of ribavirin has been 1000 to 1200 mg administered daily. This amount of ribavirin as a monotherapy has been found to be marginally effective in alleviating symptoms in a small percentage of patients, but it causes the undesirable side effect of anemia.

Currently, the approved treatment for chronic Hepatitis C Virus utilizes a combination therapy with ribavirin and interferon-alpha-2b or pegylated interferon-alpha-2b. The approved dosages of ribavirin vary from a fixed 800 mg/day up to 1200 mg/day based on body weight.

In the original clinical studies of the combination therapy of interferon alpha-2b and ribavirin REBETRON, the most notable laboratory adverse event was anemia. A reduction in hemoglobin of greater than 4 g/dl occurred in 14% of patients in the 6 month duration study and in 21% of the patients in the 12 month clinical trial. Ribavirin monotherapy also resulted in a reduction in hemoglobin of greater than 4 g/dl in 15.7% of patients with chronic hepatitis C in prior studies. The proportion of patients experiencing hemoglobin reductions of greater than 4 g/dl is similar for ribavirin mono or the combination therapy. Among the patients who started therapy with baseline hemoglobin within the normal range a reduction to below 10 g/dl was uncommon. The majority of patients had reached their lowest hemoglobin value by the first month of therapy at which time the hemoglobin stabilized or increased. Unfortunately, ribavirin dose reduction or dose cessation was necessary in certain patients because of the reductions in hemoglobin.

In a preferred embodiment, the present invention provides an improved method for treating patients having Hepatitis C Virus infection by administrating a therapeutically effective amount of an inhibitor of IMPDH such as (S)-N-3-[3-(3-methoxy-4-oxazol-5-yl-phenyl)ureido]-benzylcarbamic acid tetrahydrofuran-3-yl-ester in combination with a lower dose of ribavirin so as to allow such patients to persist in using a therapeutically effective amount of combination therapy until a sustained virologic response is achieved. Therefore, in one embodiment, the present invention provides an equivalent efficacy with a reduced side effect profile.

In one embodiment of this invention, the dosage of ribavirin is reduced from about 20% to about 90%, preferably from about 35% to about 80%, more preferably from about 50% to about 70% of the current typical dose range of 8 to 16 mg/kg/day or 600 to 1200 mg/day. In a certain embodiment of this invention, the dosage of ribavirin administered as part of the combination therapy is from about 60 to about 600 mg per day, preferably from about 100 to about 500 mg/day, and most preferably from about 100 to about 400 mg/kg a day. In another embodiment, the applicable effective dose range of ribavirin depends on the dose of the inhibitor of IMPDH. In a certain embodiment of this invention, the dose range of ribavirin depends on the sensitivity of the patient's hemoglobin levels to ribavirin administration. This daily dosage may be administered once per day in a single dose or in divided doses.

The therapeutically weight-effective amount of ribavirin administered concurrently with the pegylated interferon-alpha is from about 60 to about 600 mg per day, preferably from about 100 to about 500 mg/day, and most preferably from about 100 to about 400 mg/kg a day. The pegylated interferon-alpha dose is also preferably administered to the pediatric patient during the same period of time that such patient receives doses of ribavirin.

The term “in association with” as used herein means that ribavirin is administered as part of the combination therapy to the patient, that is, before, after or concurrently with the administration of the inhibitor of IMPDH such as (S)-N-3-[3-(3-methoxy-4-oxazol-5-yl-phenyl)ureido]-benzylcarbamic acid tetrahydrofuran-3-yl-ester. The pegylated interferon-alpha dose is preferably administered during-the same period of time that the patient receives doses of ribavirin and (S)-N-3-[3-(3-methoxy-4-oxazol-5-yl-phenyl)ureido]-benzylcarbamic acid tetrahydrofuran-3-yl-ester.

The term “inhibitors” as used herein are compounds that slow down the rate of an enzyme catalyzed reaction by binding to some form of the enzyme or enzyme substrate complex and lowering the amount of enzyme available for catalysis. The type of inhibition is determined by plotting the reciprocals of the reaction velocity (1/v) and substrate concentration (1/A) at various inhibitor concentrations. See K. M. Plowman, Enzyme Kinetics, McGraw-Hill, New York, N.Y., pp. 56-73. “Competitive inhibitors” are reversible inhibitors that affect only the slope of the plot at various inhibitor concentrations. “Uncompetitive inhibitors” are reversible inhibitors that shift the y-intercept at the various inhibitor concentrations. See K. M. Plowman, Enzyme Kinetics, McGraw-Hill, New York, N.Y., pp. 56-73.

The term “therapeutically weight-effective amount of ribavirin” means an amount that is sufficient to produce a sustained virologic response for at least about twelve weeks post treatment, preferably for at least about twenty-four weeks post treatment, most preferably forty eight weeks post treatment.

The therapeutically weight-effective amount of ribavirin administered concurrently with the pegylated interferon-alpha is from about 60 to about 600 mg per day, preferably from about 100 to about 500 mg/day, and most preferably from about 100 to about 400 mg/kg a day. The pegylated interferon-alpha dose is also preferably administered to the pediatric patient during the same period of time that such patient receives doses of ribavirin.

Interferon

Interferons are a family of naturally occurring small proteins and glycoproteins produced and secreted by most nucleated cells in response to viral infection as well as other antigenic stimuli. Interferons render cells resistant to viral infection and exhibit a wide variety of actions on cells. They exert their cellular activities by binding to specific membrane receptors on the cell surface. Once bound to the cell membrane, interferons initiate a complex sequence of intracellular events. In vitro studies demonstrated that these include the induction of various enzymes, suppression of cell proliferation, immunomodulating activities such as enhancement of the phagocytic activity of macrophages and augmentation of the specific cytotoxicity of lymphocytes for target cells, and inhibition of virus replication in virus-infected cells.

Nonimmune interferons, which include both alpha and beta interferons, are known to suppress human immunodeficiency virus (HIV) in both acutely and chronically infected cells. Poli and Fauci, 1992, AIDS Research and Human Retroviruses 8(2):191-197. Interferons, in particular, alpha interferons, have received considerable attention as therapeutic agents in the treatment of Hepatitis C Virus (HCV)-related disease due to their antiviral activity.

Interferons are known to affect a variety of cellular functions, including DNA replication and RNA and protein synthesis, in both normal and abnormal cells. Thus, cytotoxic effects of interferon are not restricted to tumor or virus infected cells but are also manifested in normal, healthy cells as well. As a result, undesirable side effects arise during interferon therapy, particularly when high doses are required. Administration of interferon can lead to myelosuppression resulting in reduced red blood cell, white blood cell and platelet levels. Higher doses of interferon commonly give rise to flu-like symptoms (e.g., fever, fatigue, headaches and chills), gastrointestinal disorders (e.g., anorexia, nausea and diarrhea), dizziness and coughing.

The term “interferon” or “IFN” as used herein means the family of highly homologous species-specific proteins that inhibit viral replication and cellular proliferation and modulate immune response. Human interferons are grouped into three classes based on their cellular origin and antigenicity: alpha-interferon (leukocytes), beta-interferon (fibroblasts) and gamma-interferon (B cells). Recombinant forms of each group have been developed and are commercially available. Subtypes in each group are based on antigenic/structural characteristics. At least 24 interferon alphas (grouped into subtypes A through H) having distinct amino acid sequences have been identified by isolating and sequencing DNA encoding these peptides. The terms “a-interferon”, “alpha interferon”, “interferon alpha” and “human leukocyte interferon” may be used interchangeably in this application to describe members of this group. Both naturally occurring and recombinant alpha-interferons, including consensus interferon, may be used in the practice-of the invention.

The purification of interferon alpha from human leukocytes isolated from the buffy coat fraction of whole blood is described in U.S. Pat. No. 4,503,035. Human leukocyte interferon prepared in this manner contains a mixture of human leukocyte interferons having different amino acid sequences. Purified natural human alpha-interferons and mixtures thereof which may be used in the practice of the invention include but are not limited to Sumiferon® interferon alpha-n1 available from Sumitomo, Japan, Wellferon® interferon alpha-n1 (Ins) available from Glaxo-Wellcome Ltd., London, Great Britain, and Alferon® interferon alpha-n3 available from the Purdue Frederick Co., Norwalk, Conn.

Typical suitable interferon-alphas include, but are not limited to, recombinant interferon alpha-2b such as Intron-A interferon available from Schering Corporation, Kenilworth, N.J., recombinant interferon alpha-2a such as Roferon interferon available from Hoffmann-La Roche, Nutley, N.J., recombinant interferon alpha-2C such as Berofor alpha 2 interferon available from Boehringer Ingelheim Pharmaceutical, Inc., Ridgefield, Conn., interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alpha-n3 a mixture of natural alpha interferons made by Interferon Sciences and available from the Purdue Frederick Co., Norwalk, Conn., under the Alferon Tradename. The use of interferon alpha-2a or alpha-2b is preferred. Since interferon alpha-2b, among all interferons, has the broadest approval throughout the world for treating chronic hepatitis C infection, it is most preferred. The manufacture of interferon alpha-2b is described in U.S. Pat. No. 4,530,901.

The advent of recombinant DNA technology applied to interferon production has permitted several human interferons to be successfully synthesized, thereby enabling the large-scale fermentation, production, isolation, and purification of various interferons to homogeneity. Recombinantly produced interferon retains its in vitro and in vivo antiviral and immunomodulatory activities. It is also understood that the recombinant techniques could also include a glycosylation site for addition of a carbohydrate moiety on the recombinantly-derived polypeptide.

The construction of recombinant DNA plasmids containing sequences encoding at least part of human leukocyte interferon and the expression in E. coli of a polypeptide having immunological or biological activity of human leukocyte interferon is disclosed in U.S. Pat. No. 4,530,901 and European Patent No. EP 0 032 134. The construction of hybrid alpha-interferon genes containing combinations of different subtype sequences (e.g., A and D, A and B, A and F) is disclosed in U.S. Pat. Nos. 4,414,150, 4,456,748 and 4,678,751.

U.S. Pat. Nos. 4,695,623 and 4,897,471 disclose human leukocyte interferon polypeptides, referred to as consensus interferon, which have amino acid sequences that include common or predominant amino acids found in each position among naturally occurring interferon alpha subtype polypeptides.

Interferon alpha-2b has been shown to be safe and effective when administered subcutaneously at a dose of 3×10⁶ international units (IU) three times a week for 24 weeks for the treatment of chronic hepatitis [C. Causse et al., Gastroenterology 101:497-502 (1991); Davis et al., New Eng. J. Med. 321:1501-1506 (1991); Marcellin et al., Hepatology, 13(3):393-393 (1991). This amount and duration alleviates symptoms of hepatitis C and biochemical or histological evidence of ongoing inflammation of the liver in some patients but it also causes undesirable side effects, e.g., flu-like symptoms. Thus, thrice weekly injections place a burden on the patient and have a significant impact on the patient's quality of life.

In prior treatment of chronic hepatitis C infection with alpha-interferon monotherapy, alpha-interferon has been administered in dosages of about 3 to about 10 million International units (IU) thrice weekly. Alternatively 3 to 10 million IU of alpha-interferon has been administered QOD (every other day) or daily. The duration of the prior dosages has been from 12 to 24 months. This amount and duration of alpha-interferon monotherapy alleviates symptoms of hepatitis C in some of the patients, but it causes undesirable side effects, e.g. flu-like symptoms, in some.

Pegylated Interferon

The anti-HCV drugs of this invention may also be administered in association with pegylated interferon alpha as part of any anti-HCV drug therapy.

The term “pegylated interferon alpha” refers to interferon alpha covalently attached to or conjugated to polyethylene glycol, preferably interferon alpha-2a and -2b. The preferred polyethylene-glycol-interferon alpha-2b conjugate is PEG₁₂₀₀₀-interferon alpha-2b. The phrases “12,000 molecular weight polyethylene glycol conjugated interferon alpha” and “PEG₁₂₀₀₀-IFN alpha” as used herein mean conjugates such as those prepared according to the methods of International Application No. WO 95/13090 and containing urethane linkages between the interferon alpha-2a or -2b amino groups and polyethylene glycol having an average molecular weight of 12000.

As used herein, “μg/kg of PEG₁₂₀₀₀-IFN alpha-2b” indicate the number of micrograms of protein in the conjugate per kg of bodyweight of the patient and not the combined microgram quantity of the protein and the conjugate.

The preferred PEG₁₂₀₀₀-interferon alpha-2b (IFN alpha-2b) is prepared by attaching a PEG polymer to the epsilon amino group of a lysine residue in the IFN alpha-2b molecule. A single PEG₁₂₀₀₀ molecule is conjugated to free amino groups on an IFN alpha-2b molecule via a urethane linkage. This conjugate is characterized by the molecular weight of PEG₁₂₀₀₀ attached. The PEG₁₂₀₀₀-IFN alpha-2b conjugate is formulated as a lyophilized powder for injection. The objective of conjugation of alpha interferon with PEG is to improve the delivery of the protein by significantly prolonging its plasma half-life, and thereby provide protracted activity of alpha interferon.

Other interferon alpha conjugates can be prepared by coupling an interferon alpha to a water-soluble polymer. A non-limiting list of such polymers includes other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described in U.S. Pat. No. 4,766,106, U.S. Pat. No. 4,917,888, European Patent Application No. 0 236 987, European Patent Application Nos. 0510 356, 0 593 868 and 0 809 996 (pegylated interferon alpha-2a) and International Publication No. WO 95/13090.

Pharmaceutical compositions of pegylated interferon alpha suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g. NaCl), preservatives (e.g. thimerosol, cresol or benzylalcohol), and surfactants (e.g. tween or polysorabates) in sterile water for injection. The pegylated interferon alpha-may be stored as lyophilized powders under refrigeration at 2°-8° C. The reconstituted aqueous solutions are stable when stored between 2° and 8° C. and used within 24 hours of reconstitution. See for example U.S. Pat. Nos. 4,492,537; 5,762,923 and 5,766,582. The reconstituted aqueous solutions may also be stored in prefilled, multi-dose syringes such as those useful for delivery of drugs such as insulin. Typical suitable syringes include systems comprising a prefilled vial attached to a pen-type syringe such as the NOVOLET Novo Pen available from Novo Nordisk, as well as prefilled, pen-type syringes that allow easy self-injection by the user. Other syringe systems include a pen-type syringe comprising a glass cartridge containing a diluent and lyophilized pegylated interferon alpha powder in a separate compartment.

In the practice of the invention, the preferred PEG₁₂₀₀₀-IFN alpha-2a or -2b conjugates may be administered to patients infected with the Hepatitis C Virus. Use of PEG₁₂₀₀₀-IFN alpha-2b is preferred.

In previous studies, three doses of PEG₁₂₀₀₀-IFN alpha-2b (0.5, 1.0, 1.5 μg/kg) administered once a week were found to have equal to or better antiviral activity than the interferon alpha control 3 MIU TIW (measured by loss of HCVRNA (PCR)) at 4, 8 and 12 weeks of therapy. See U.S. Pat. No. 6,177,074 to Glue et al. (2001), the disclosure of which is hereby incorporated by reference in its entirety.

The amount of the PEG₁₂₀₀₀-IFN alpha conjugate administered to treat any of the conditions described above is based on the IFN alpha activity of the polymeric conjugate. It is an amount that is sufficient to significantly affect a positive clinical response while maintaining diminished side effects. The amount of PEG₁₂₀₀₀-IFN alpha-2b, which may be administered, is in the range of at least about 0.25 μg/kg in single or divided doses. In more preferred embodiments, the amount administered is in the range of about 0.25-2.5 μg/kg, or 0.5-1.5 μg/kg in single or divided doses.

As used herein, dosages measured in μg/kg of PEG₁₂₀₀₀-IFN alpha-2b indicate the number of micrograms of the active constituent, i.e., interferon alpha, of the interferon-polyethylene glycol conjugate to be administered per kilogram weight of the patient. This quantity is independent of the molecular weight of the polyethylene glycol in the conjugate. Thus, a defined microgram quantity of a protein polyethylene glycol conjugate reflects the microgram quantity of protein in the conjugate, and not the combined microgram quantity of the protein and the conjugate.

Administration of the described dosages may be every other day, but is preferably once or twice a week. Doses are administered over at least a 24 week period by injection.

Administration of the dose can be intravenous, subcutaneous, intramuscular, or any other acceptable systemic method. Based on the judgment of the attending clinician, the amount of drug administered and the treatment regimen used will, of course, be dependent on the age, sex and medical history of the patient being treated, the neutrophil count (e.g. the severity of the neutropenia), the severity of the specific disease condition and the tolerance of the patient to the treatment as evidenced by local toxicity and by systemic side-effects. Dosage amount and frequency may be determined during initial screenings of neutrophil count.

For any route of administration, divided or single doses may be used. For example, when a subcutaneous injection is used to deliver, for example, 1.5 μg/kg of PEG₁₂₀₀₀-IFN alpha-2b over one week, two injections of 0.75 μg/kg at 0 and 72 hours may be administered.

Treatable Conditions

Conditions that can be treated in accordance with the present invention are generally those infections that are susceptible to treatment with interferon alpha. For example, susceptible conditions include conditions, which would respond positively or favorably as these terms are known in the medical arts to interferon alpha-based therapy. For purposes of the invention, conditions that can be treated with interferon alpha therapy include those conditions in which treatment with an interferon alpha shows some efficacy, but which may not be treatable with interferon alpha because the negative side effects outweigh the benefits of the treatment. For example, side effects accompanying interferon alpha therapy have virtually ruled out treatment of Epstein Barr virus using interferon alpha.

Combination Therapy

The effectiveness of treatment may be determined by controlled clinical trials of the combination therapy versus monotherapy. The efficacy of the combination therapy in alleviating the signs and symptoms of chronic hepatitis C infection and the frequency and severity of the side effects will be compared with previous alpha-interferon and ribavirin monotherapy and previous alpha-interferon/ribavirin combination therapies. Three populations suffering from chronic hepatitis C infection will be evaluated:

1. Patients previously untreated.

2. Patients previously treated with interferon or ribavirin monotherapy or interferon/ribavirin combination therapy and who had subsequently relapsed.

3. Patients who were non-responsive to previous treatment with interferon and/or ribavirin.

4. Patients who discontinued combination therapy due to side effects.

The effectiveness of the combination therapy will be determined by the extent to which the previously described signs and symptoms of chronic hepatitis are alleviated and the extent to which the normal side effects of alpha-interferon and ribavirin are eliminated or substantially reduced. The reduction or elimination of side effects will be accomplished by the ability to use reduced dosage or increased dosage duration or both for interferon and/or ribavirin compared to the previous monotherapies.

To practice one embodiment of the invention, alpha interferon and combinations of a ribonucleoside analog and an inhibitor of an enzyme controlling GTP pools are administered to the patient exhibiting one or more of the above signs or symptoms in amounts sufficient to eliminate or at least alleviate one or more of the signs or symptoms.

To practice a particular embodiment of the invention, alpha interferon and combinations of ribavirin and an IMPDH inhibitor are administered to the patient exhibiting one or more of the above signs or symptoms in amounts sufficient to eliminate or at least alleviate one or more of the signs or symptoms.

To practice the invention, the therapeutically effective amount of the combination therapy of (S)-N-3-[3-(3-methoxy-4-oxazol-5-yl-phenyl)ureido]-benzylcarbamic acid tetrahydrofuran-3-yl-ester, ribavirin and interferon alpha-2b is administered to the patient exhibiting one or more of the above signs or symptoms in amounts sufficient to eliminate or at least alleviate one or more of the signs or symptoms.

Ribavirin and an inhibitor of IMPDH such as (S)-N-3-[3-(3-methoxy-4-oxazol-5-yl-phenyl)ureido]-benzylcarbamic acid tetrahydrofuran-3-yl-ester are administered to the patient in association with interferon-alpha, that is, the interferon-alpha dose is administered during the same period of time that the patient receives doses of Ribavirin and the inhibitor of IMPDH such as (S)-N-3-[3-(3-methoxy-4-oxazol-5-yl-phenyl)ureido]-benzylcarbamic acid tetrahydrofuran-3-yl-ester of the present invention. Most interferon-alpha formulations are not effective when administered orally, so the preferred method for administering the interferon-alpha is parenterally, preferably by subcutaneous, IV, or IM, injection. Ribavirin and the uncompetitive inhibitor of IMPDH, such as (S)-N-3-[3-(3-methoxy-4-oxazol-5-yl-phenyl)ureido]-benzylcarbamic acid tetrahydrofuran-3-yl-ester may be administered orally in capsule or tablet form in association with the parenteral administration of interferon-alpha. Of course, other types of administration of both medicaments, as they become available are contemplated, such as by nasal spray, transdermally, by suppository, by sustained release dosage form, etc. Any form of administration will work so long as the proper dosages are delivered without destroying the active ingredient.

In order to follow the course of HCV replication in subjects in response to drug treatment, HCV RNA may be measured in serum samples by, for example, a nested polymerase chain reaction assay that uses two sets of primers derived from the NS3 and NS4 non-structural gene regions of the HCV genome. See Farci et al., New Eng. J. Med. 325:98-104 (1991) and Ulrich et al., J. Clin. Invest. 86:1609-1614.

Antiviral activity may be measured by changes in HCV-RNA titer. HCV RNA data may be analyzed by comparing titers at the end of treatment with a pre-treatment baseline measurement. Reduction in HCV RNA by week 4 provides evidence of antiviral activity of a treatment. Kleter et al., Antimicrob. Agents Chemother. 37(3):595-97 (1993); Orito et al., J. Medical Virology, 46:109-115 (1995). Changes of at least two orders of magnitude (>2 log) is interpreted as evidence of antiviral activity.

Safety and tolerability may be determined by clinical evaluations and periodic monitoring of hematological parameters such as white blood cell, hemoglobin, red blood cell, platelet and neutrophil counts.

Pharmaceutical Compositions

The pharmaceutical compositions of ribavirin and an uncompetitive IMPDH inhibitor may be adapted for any mode of administration e.g., for oral, parenteral, e.g., subcutaneous (SC), intramuscular (IM), intravenous (IV) and intraperitoneal (IP), topical or vaginal administration or by inhalation (orally or intranasally). Preferably, the pharmaceutical composition of ribavirin and an uncompetitive IMPDH inhibitor is administered orally.

Such compositions may be formulated by combining ribavirin and uncompetitive IMPDH inhibitor or equivalent amounts of pharmaceutically acceptable salts thereof with a suitable, inert, pharmaceutically acceptable carrier or diluent which may be either solid or liquid. Ribavirin and an IMPDH inhibitor may be converted into the pharmaceutically acceptable acid addition salts by admixing them with equivalent amounts of the pharmaceutically acceptable acids. Typically suitable pharmaceutically acceptable acids include the mineral acids, e.g., HNO₃H₂SO₄, H₃PO₄, HCl, HBr, organic acids, including, but not limited to, acetic, trifluoroacetic, propionic, lactic, maleic, succinic, tartaric, glucuronic and citric acids as well as alkyl or arylsulfonic acids, such as p-toluenesulfonic acid, 2-naphthalenesulfonic acid, or methanesulfonic acid. The preferred pharmaceutically acceptable salts are trifluoroacetate, tosylate, mesylate, and chloride.

Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1999), Mack Publishing Co., Easton, Pa.

Liquid form preparations include solutions, suspensions and emulsions. For example, water or water-propylene glycol solutions may be used for parenteral injection. Solid form preparations may be converted into liquid preparations shortly before use for either oral or parenteral administration. Parenteral forms may be injected intravenously, intramuscularly or subcutaneously and are usually in the form of sterile solutions and may contain tonicity agents (salts or glucose), and buffers. Opacifiers may be included in oral solutions, suspensions and emulsions. Liquid form preparations may also include solutions for intranasal administration.

In one embodiment, the pharmaceutical compositions of this invention also comprise a carrier, wherein the carrier is in a liquid form and the liquid form is selected from the group consisting of an aqueous solution, an alcohol solution, an emulsion, a suspension, a suspension reconstituted from non-effervescent or effervescent preparations, and a suspension in pharmaceutically acceptable fats or oils. Alternatively, the liquid form further comprises a member selected from the group consisting of a suspending agent, a diluent, a sweetener, a flavorant, a colorant, a preservative, an emulsifying agent, a coloring agent, and mixtures thereof.

Aerosol preparations suitable for inhalation may include solutions and solids in powder form, which may be in combination with a pharmaceutically acceptable carrier, such as an inert compressed gas, e.g., nitrogen.

Also included are solid form preparations, which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in a unit dosage form. In such form, the preparation is subdivided into suitably sized unit doses containing appropriate quantities of the active component, e.g., an effective amount to achieve the desired purpose.

The following examples are offered to illustrate the invention and are not intended to limit it in any way.

### EXAMPLES

We examined the mutagenic effect of ribavirin and other IMPDH inhibitors on HCV using the recently developed HCV replicon system. See German Patent No. DE19915178A1 to Bartenschlager, the disclosure of which is hereby incorporated by reference in its entirety. In the replicon system, an autonomously replicating clone of HCV comprising HCV 5′-NTR (NTR non translated region), NS3, NS4A, NS4B, NS5A, NS5B and a neomycin selectable marker gene that is linked to the HCV genes is inserted into human hepatoma cell line known as Huh-7 cells. The HCV-RNAs produced by the autonomously replicating clone of HCV in the Huh-7 cells are extracted and retransfected into naïve Huh-7 cells to evaluate the replication competence and efficiency of the HCV-RNA. Therefore, the effect of anti-viral treatments on the HCV-RNAs produced may be evaluated by growing the replicon cells in the presence or absence of such anti-viral treatments, then extracting the replicon RNA from treated cells and transfecting it into naive Huh 7 cells and measuring colony formation. The effect of the combination of ribavirin, and IMPDH inhibitors on HCV replication in vitro was evaluated using the replicon system as described below.

Compounds. Ribavirin (1-beta-D-ribofuranosyl-1,2,4-trizole-3-carboximide) and the IMPDH inhibitor IMPDH-I (See Table 1), which corresponds to the compound (S)-N-3-[3-(3-methoxy-4-oxazol-5-yl-phenyl)ureido]-benzylcarbamic acid tetrahydrofuran-3-yl-ester were obtained from Schering-Plough Research Institute. Ribavirin was stored frozen as a 20 mM stock solution in phosphate buffered saline (PBS). IMPDH-I was stored as a 20 mM stock solution in dimethyl sulfoxide (DMSO). Mycophenolic acid (MPA) and guanosine were both purchased from Sigma (catalog No. M3536 and G6264, respectively) and were stored frozen in DMSO as 20 mM stock solutions. Interferon (IFN) alpha-2b (Intron A) was obtained from Schering-Plough and was stored frozen in water as 10⁶ IU/ml stock solution.

HCV Cell Culture System. Human hepatoma cell line Huh-7 cells were grown in Dulbecco's modified minimal essential medium (DMEM, Cellgro) supplemented with 10% fetal bovine serum, 4 mM L-glutamine, non-essential amino acids, 10 mM HEPES, 0.075% sodium bicarbonate, 100 U/ml penicillin and 100 ug/ml streptomycin, and 1 mM sodium pyruvate. HCV subgenomic replicon (clone 16, containing NS5A S1I791 adaptive mutation as described in Blight et al., Science 290:1972 (2000)) was constructed by Schering-Plough Research Institute as described by R. Bartenschlager. See Lohmann, et al., Science 285:5424 (2000). The replicon-bearing Huh-7 cells were maintained in the same media as described above with 0.5 mg/ml G418 (Geneticin, Gibco-BRL).

HCV RNA Replicon Copy Number measurement by Taqman assays. Replicon cells were seeded at 3000 cells per well in 96-well Bio-coated plates (Becton Dickinson) and allowed to adhere overnight. The next day the culture medium was replaced with 5% serum medium containing the experimental compound. After 72 hours the plate was harvested by aspirating medium, washing with PBS twice, and putting in 30 111 cell lysis buffer (Ambion, catalog No. 8722). The plate was then heated at 75° C. for 5 min followed by freezing at −70° C. for 10 min. The cell lysate was then measured for HCV replicon copy number by one-step real-time RT-PCR (Taqman assay) using an ABI 7700 instrument. The primers, which correspond to HCV NS5B, are: forward, 5′-ATGGACAGGCGCCCTGA-3′ (SEQ ID NO: 1), and reverse, 5′-TTGATGGGCAGCTTGGTTTC-3′ (SEQ ID NO: 2). The double fluorescence-labeled probe (5′-CACGCCATGCGCTGCGG-3′) (SEQ ID NO: 3) was purchased from Applied Biosystems (part No. P6448-6464). The cellular GAPDH (glyceraldehyde 3-phosphate dehydrogenase) mRNA from the same cell lysate was used as an internal control for cell number and metabolic status (primers/probes purchased from Perkin Elmer, part No. 4310859). Every experiment was performed in triplicate. The entire study was conducted independently two times.

Cytotoxicity determination. Cytotoxicity of a given drug or combination of drugs was determined by two methods: the MTS assay using the compound 3-(4,5-dimethylthiazol-2-yl)-5-(3-carboxymethoxyphenyl)-2-(4-sulfophenyl)-2-(4-sulfophenyl)-2H-terrazolium, inner salt and cellular GAPDH RNA quantification. The MTS assay was performed according to manufacturer's instruction (CellTiter 96 Aqueous One Solution cell proliferation assay, Promega, Part No. TB245). Cellular GAPDH RNA was measured by real-time Taqman RT-PCR as described above.

Specific Colony Formation Efficiency (SCFE) determination. Replicon cells were loaded with compound(s) for 10 days under complete DMEM (without G418), with medium changes every 2-3 days. The total cytoplasmic RNA was extracted using RNA Easy kit from Qiagen (catalog No. 74104) and the RNA concentration was determined by absorbance at 260 nm. HCV replicon copy number in each RNA sample was determined by Taqman assay using a standard curve. For electroporation of RNA obtained from compound-treated replicon cells, 8 μg of total RNA suspended in 50 μl PBS was mixed with 50 μl suspension of 2×10⁶ Huh-7 cells in a 0.1 cm cuvette (Bio-Rad, catalog No. 165-2089). As a control, wild type replicon RNA was serially diluted with total RNA from naïve Huh-7 cells. After two pulses at 960 μF, 350 Volts using a Gene Pulser system (Bio-Rad), cells were immediately transferred to 10 ml of complete DMEM medium (without G418) and seeded onto a 10-cm culture dish. After 24 h, medium was replaced by complete DMEM supplemented with 0.5 mg/ml G418. About three weeks later, colonies were counted by staining with crystal violet (0.5 g/liter in 30% methanol, 3.2% formaldehyde). Representative results of multiple (>2) independent transfections are shown.

TABLE 1
Effect of Anti-Viral Treatments on HCV Replicon RNA

| Example | | ¹Replicon | No. of | |
|---|---|---|---|---|
| Number | Description | Copy No. | Colonies | ²SCFE |
| 1 | Control - no drug | 10 | 740 | 740 |
| 2 | 10 μM ribavirin | 14 | 650 | 480 |
| 3 | 50 μM ribavirin | 12 | 420 | 360 |
| 4 | 100 μM ribavirin | 6 | 110 | 200 |
| 5 | 100 nM IMPDH-I³ | 10 | 370 | 360 |
| 6 | Control - no replicon and | 0 | 0 | 0 |
| | no drug | | | |
| 7 | 10 μM ribavirin + | 10 | 210 | 210 |
| | 100 nM IMPDH-I³ | | | |
| 8 | 50 μM ribavirin + | 6 | 36 | 60 |
| | 100 nM IMPDH-I | | | |
| 9 | 100 μM ribavirin + | 3 | 7 | 30 |
| | 100 nM IMPDH-I | | | |
| 10 | 10 μM ribavirin + | 6 | 740 | 1200 |
| | 100 nM IMPDH-I + | | | |
| | 10 μM Guan.⁴ | | | |
| 11 | 50 μM ribavirin + | 6 | 540 | 840 |
| | 100 nM IMPDH-I + | | | |
| | 10 μM Guan. | | | |
| 12 | 100 μM ribavirin + | 5 | 270 | 510 |
| | 100 nM IMPDH-I + | | | |
| | 10 μM Guan. | | | |
| 13 | 10 μM ribavirin + | 8 | 160 | 200 |
| | 2 μM MPA⁵ | | | |
| 14 | 100 μM ribavirin + | 4 | 20 | 40 |
| | 2 μM MPA | | | |
| 15 | 10 μM ribavirin + | 6 | 480 | 750 |
| | 2 μM MPA + | | | |
| | 10 μM Guan. | | | |
| 16 | 100 μM ribavirin + | 5 | 210 | 420 |
| | 2 μM MPA + | | | |
| | 10 μM Guan. | | | |
| 17 | 2 μM MPA | 9 | 470 | 510 |
| 18 | 10 μM Guan. | 12 | 210 | 180 |
| 19 | 100 nM IMPDH-I + | 8 | 850 | 1100 |
| | 10 μM Guan. | | | |
| 20 | 10 μM ribavirin + | 12 | 70 | 60 |
| | 10 μM Guan. | | | |
| 21 | 50 μM ribavirin + | 10 | 120 | 120 |
| | 10 μM Guan. | | | |
| 22 | 100 μM ribavirin + | 8 | 350 | 430 |
| | 10 μM Guan. | | | |

¹Replicon copies X 10⁷/8 μg total RNA
²SCFE refers to the Specific Colony Formation Efficiency (colonies/10⁸ HCV RNA genomes).
³IMPDH-I refers to an inhibitor of IMPDH, in these examples it refers to the compound (S)-N-3-[3-(3-methoxy-4-oxazol-5-yl-phenyl)ureido]-benzylcarbamic acid tetrahydrofuran-3-yl-ester.
⁴Guan. is guanosine
⁵MPA refers to mycophenolic acid

Ribavirin is clinically administered in combination with interferon-alpha (IFN-α) or PEGylated IFN-alpha to treat chronic Hepatitis C. It has been shown by in vitro biochemical assays that ribavirin triphosphate (RTP) competes with cellular guanosine triphosphate (GTP) for incorporation into the newly synthesized viral genomes. Incorporated ribavirin templates either uridine monophosphate (UMP) or cytidine monophosphate (CMP) with equal efficiency, thereby inducing mutations in the viral genomes. Accumulation of mutations may push the virus beyond the tolerable set points in its mutation rate, leading to an overall reduced fitness of the viral population.

In Table 1, Examples 1-4, we determined the effect of increasing concentrations of ribavirin on the fitness of the replicon produced. As shown in Table 1, Examples 1-4, Ribavirin alone was sufficient to cause a significant reduction in the fitness of the replicon (˜4 fold with 100 μM ribavirin) as indicated by the decrease in the viral genome's specific colony formation efficiency.

The compound (S)-N-3-[3-(3-methoxy-4-oxazol-5-yl-phenyl)ureido]-benzylcarbamic acid tetrahydrofuran-3-yl-ester, known as IMPDH-I is a potent inosine monophosphate dehydrogenase (IMPDH) inhibitor (Kᵢ˜10 nM) which blocks the de novo synthesis of guanosine monophosphate (GMP). See J. Jain et al., Journal Pharmaceutical Sciences 90: 625-637 (2001). In Table 1, Examples 5 through 12, we evaluated whether the mutagenic effect of ribavirin on the HCV replicon can be potentiated by co-administration of IMPDH-I. As shown in Table 1, Example 7-9 the use of 10 μM ribavirin in combination with 100 nM IMPDH-I produced an approximate 4 fold reduction in the fitness of the replicon. The combination of 100 μM ribavirin together with 100 nM IMPDH-I produced an approximately 25 fold reduction in the fitness of the replicon (Example 9).

Next, we determined whether the potentiation of ribavirin's effect was due to a lowering of the GTP pools by IMPDH-I. We found that the combined mutagenic effect was significantly reversed by addition of exogenous guanosine (10 μM). See Table 1, Examples 10-12. These results suggest that GTP pool reduction was involved in the anti-HCV effect from co-administration of ribavirin and IMPDH-I. The administration of 100 nM IMPDH-I alone had only a marginal effect on the replicon. See Table 1, Example 5.

A similar synergistic effect was seen with the combination treatment of ribavirin and another potent IMPDH inhibitor, mycophenolic acid (MPA). See Table 1, Examples 13-22. In these examples, 2 μM mycophenolic acid was combined with either 10 μM or 100 μM ribavirin treatments. See Examples 13-14. The combination of 100 μM ribavirin and 2 μM mycophenolic acid produced a more than 18 fold reduction in specific colony formation efficiency over the no drug control (Example 14).

In Table 1, Examples 15-16 we found the anti-replicon effect of ribavirin and mycophenolic acid could be largely suppressed by adding exogenous guanosine (10 μM) suggesting that GTP pools were lowered by mycophenolic acid.

In summary, the above results support the notion that ribavirin is an HCV mutagen and that its mutagenic effect can be enhanced by combining it with IMPDH inhibitors such as the compound (S)-N-3-[3-(3-methoxy-4-oxazol-5-yl-phenyl)ureido]-benzylcarbamic acid tetrahydrofuran-3-yl-ester (IMPDH-I) and mycophenolic acid.

The use of ribavirin in combination with the compound (S)-N-3-[3-(3-methoxy-4-oxazol-5-yl-phenyl)ureido]-benzylcarbamic acid tetrahydrofuran-3-yl-ester (IMPDH-I) for treating viral infections produces the following advantages over the current therapies. First, ribavirin and IMPDH-I combination therapy may be used to achieve the same antiviral efficacy with a lower dose of ribavirin (10 μM ribavirin in combination with 100 nM IMPDH-I is similar to 100 μM ribavirin alone) (Comparing Example 4 and 7). In this way, patients who cannot tolerate the side effects of 100 μM ribavirin, such as hemolytic anemia, may still obtain the same level of benefit. Alternatively, in cases where the side effects of ribavirin are not the limiting factor, a higher efficacy of treatment can be achieved with the same ribavirin dosage by adding 100 nM IMPDH-I to the treatment (Comparing 100 μM ribavirin alone to 100 μM ribavirin and 100 nM IMPDH-I). See Table 1, Examples 4 and 9).

### Example 23

### West Nile Virus

The effects of combining ribavirin and IMPDH inhibitors on West Nile Virus replication is evaluated as follows:

A full length cDNA clone of WNV is constructed by assembling fragments generated using RT-PCR from the RNA genome as described by P. Y. Shi et al., J. Virology, 76:5847-5856 (2002), the disclosure of which is hereby incorporated by reference in its entirety. Next, the WNV cDNA is cloned into plasmid pBR322 and positioned under the control of the T7 promoter. The plasmid is amplified by growth in Eschericha coli HB 101.

Vero cells (ATCC CCL-81) are grown in minimum essential medium (MEM) with 10% fetal bovine serum (FBS), 10 U/ml penicillin and 10 micrograms/ml streptomycin. BHK-21 cells are grown in Dulbecco's modification of minimum essential medium (MEM) with 10% fetal bovine serum (FBS), 0.1 mM nonessential amino acids, 10 U/ml penicillin and 10 micrograms/ml streptomycin. All cells are grown and maintained as described by P. Y. Shi et al., J. Virology, 76:5847-5856 (2002), the disclosure of which is hereby incorporated by reference in its entirety.

TABLE 2
Effect of Anti-Viral Treatments on West Nile Virus Replication

| Number | Vero Cells |
|---|---|
| 1 | Control - no drug |
| 2 | 10 μM ribavirin |
| 3 | 50 μM ribavirin |
| 4 | 100 μM ribavirin |
| 5 | 100 nM IMPDH-I³ |
| 6 | Control - no virus and no drug |
| 7 | 10 μM ribavirin + 100 nM IMPDH-I¹ |
| 8 | 50 μM ribavirin + 100 nM IMPDH-I |
| 9 | 100 μM ribavirin + 100 nM IMPDH-I |
| 10 | 10 μM ribavirin + 100 nM IMPDH-I + 10 μM Guan.² |
| 11 | 50 μM ribavirin + 100 nM IMPDH-I + 10 μM Guan. |
| 12 | 100 μM ribavirin + 100 nM IMPDH-I + 10 μM Guan. |
| 13 | 10 μM ribavirin + 2 μM MPA³ |
| 14 | 100 μM ribavirin + 2 μM MPA |
| 15 | 10 μM ribavirin + 2 μM MPA + 10 μM Guan. |
| 16 | 100 μM ribavirin + 2 μM MPA + 10 μM Guan. |
| 17 | 2 μM MPA |
| 18 | 10 μM Guan. |
| 19 | 100 nM IMPDH-I + 10 μM Guan. |
| 20 | 10 μM ribavirin + 10 μM Guan. |
| 21 | 50 μM ribavirin + 10 μM Guan. |
| 22 | 100 μM ribavirin + 10 μM Guan. |

¹IMPDH-I refers to IMPDH inhibitor
²Guan. refers to guanosine
³MPA refers to mycophenolic acid

Infectious virus is generated through in vitro transcription of the WNV cDNA as follows. First, the plasmid containing the WNV cDNA clone is linearized with an appropriate restriction enzyme for use as a template in run-off in vitro transcription reactions to generate infectious RNA. Following phenol-chloroform and ether extractions the template WNV cDNA is transcribed essentially as described using T7 RNA polymerase. See N. L. Davis et al., Virology 171:189-204 (1989) and R. C. Rice et al., J. Virol. 61:3809-3819 (1987). The transcription products are used to transfect BHK-21 cells. Infectious virus is collected from BHK-21 cell supernatants, purified and titered.

The effect of various antiviral therapies on the replication capacity of WNV is determined as follows: Separate cultures of vero cells are maintained in growth medium supplemented with the antiviral compounds indicated in Table 2 above. The cells are pretreated prior to infection in growth medium supplemented with the indicated concentrations of antiviral compounds under 5% CO₂ and at 37 degrees C. After three days subconfluent cells are infected at a multiplicity of infection (MOI) of 5 in triplicate wells with the virus stock and maintained in growth medium supplemented with the antiviral compounds and incubated under 5% CO₂ and at 37 degrees C. for at least 124 hours. Aliquots of medium are removed at 12-24 hour intervals and stored at −70 degrees C. Harvested virus is used in plaque assays on BHK-21 cells to compare the replication capacity of the viruses grown on the various treatments shown in Table 2. See M. J. Pryor et al., J. Gen Virol. 79:2631-2639 (1998); P. Y. Shi et al., J. Virology, 76:5847-5856 (2002) and R. C. Gualano et al., J. Gen. Virol. 79:437-446 (1998), the disclosures of which are hereby incorporated by reference in their entirety.

### Example 24

### Dengue Virus

The effects of combining ribavirin and IMPDH inhibitors on Dengue Virus replication is evaluated as follows:

BHK-21 cells are maintained in a modified eagle modification of minimum essential medium (MEM) containing Hank's salt solution with 10% fetal bovine serum (FBS), 0.1 mM nonessential amino acids, 100 U/ml penicillin, 100 micrograms/ml streptomycin and are incubated under 5% CO₂ and at 37 degrees C. The cells are grown and maintained as described by B. Malewicz and H. M. Jenkin, J. Clin. Microbiol., 9:609-614 (1979), the disclosure of which is hereby incorporated by reference in its entirety.

The effect of various antiviral therapies on the replication capacity of DV is determined as follows. Dengue Virus types 1, 2 & 4 are obtained as 10% mouse brain homogenates as described by B. Malewicz and H. M. Jenkin, J. Clin. Microbiol., 9:609-614 (1979), the disclosure of which is hereby incorporated by reference in its entirety. Virus stocks are prepared by growth on BHK-21 cells of virus from mouse brain homogenates. Infectious virus is collected from BHK-21 cell supernatants, purified and titered. Next, separate cultures of BHK-21 cells are maintained in growth medium and supplemented with the concentrations of antiviral compounds indicated in Table 2 above. The cells are pretreated in growth medium supplemented with these compounds and under 5% CO₂ and at 37 degrees C. After three days, subconfluent cells are infected at a multiplicity of infection (MOI) of 5 in triplicate wells with the virus stock. Next, the infected cells are incubated in growth medium supplemented with the antiviral compounds indicated in Table 2 and under 5% CO₂ and at 37 degrees C. for up to 124 hours. Aliquots of medium are removed at 12-24 hour intervals and stored at −70 degrees C. Harvested virus is used in plaque assays on BHK-21 cells to compare the replication capacity of the viruses grown on the various treatments shown in Table 2. See M. J. Pryor et al., J. Gen Virol. 79:2631-2639 (1998); P. Y. Shi et al., J. Virology, 76:5847-5856 (2002) and R. C. Gualano et al., J. Gen. Virol. 79:437-446 (1998), the disclosures of which are hereby incorporated by reference in their entirety.

### Example 25

### Venezuelan Equine Encephalitis Virus

The effects of combining ribavirin and IMPDH inhibitors on Venezuelan Equine Encephalitis Virus replication is evaluated as follows:

A full length cDNA clone of Venezuelan Equine Encephalitis Virus is constructed by assembling fragments generated using RT-PCR from the RNA genome as described by N. L. Davis et al., Virology 171:189-204 (1989), the disclosure of which is hereby incorporated by reference in its entirety. Next, the VEEV cDNA is cloned into plasmid pBR322 with the multiple cloning region of pUC 118 and positioned under the control of the T7 promoter. The plasmid is amplified by growth in Eschericha coli HB101.

BHK-21 cells are maintained in a modified eagle modification of minimum essential medium (MEM) containing 10% tryptose phosphate broth, 5% fetal bovine serum (FBS), 0.1 mM nonessential amino acids, 25 μg gentamycin sulfate and are incubated under 5% CO₂ and at 37 degrees C. Cells are grown and maintained as described by N. L. Davis et al., Virology 171:189-204 (1989), the disclosure of which is hereby incorporated by reference in its entirety.

Infectious virus is generated through in vitro transcription of the VEEV cDNA as follows. First, the plasmid containing the VEEV cDNA clone is linearized with an appropriate restriction enzyme for use as a template in run-off in vitro transcription reactions to generate infectious RNA. Following phenol-chloroform and ether extractions the template VEEV cDNA is transcribed essentially as described using T7 RNA polymerase. See N. L. Davis et al., Virology 171:189-204 (1989) and R. C. Rice et al., J. Virol. 61:3809-3819 (1987). The transcription products are used to transfect BHK-21 cells. Infectious virus is collected from BHK-21 cell supernatants, purified and titered.

Next, separate cultures of vero cells are maintained in growth medium supplemented with the concentrations of antiviral compounds indicated in Table 2 above. The cells are pretreated prior to infection in growth medium supplemented with antiviral compounds and under 5% CO₂ and at 37 degrees C. After three days subconfluent cells are infected at a multiplicity of infection (MOI) of 5 in triplicate wells with the virus stock. Next, the infected cells are maintained in growth medium supplemented with the antiviral compounds and incubated under 5% CO₂ and at 37 degrees C. for up to 124 hours. Aliquots of medium are removed at 12-24 hour intervals and stored at −70 degrees C. Harvested virus is used to perform plaque assays on BHK-21 cells to compare the replication capacity of the viruses grown on the various treatments shown in Table 2 as described. See M. J. Pryor et al., J. Gen Virol. 79:2631-2639 (1998); P. Y. Shi et al., J. Virology, 76:5847-5856 (2002) and R. C. Gualano et al., J. Gen. Virol. 79:437-446 (1998), the disclosures of which are hereby incorporated by reference in their entirety.

### Example 26

### Yellow Fever Virus

The effects of combining ribavirin and IMPDH inhibitors on Yellow Fever Virus (YFV) replication is evaluated as follows:

Vero cells (ATCC CCL-81) are grown in minimum essential medium (MEM) with 10% fetal bovine serum (FBS), 10 U/ml penicillin and 10 micrograms/ml streptomycin. BHK-21 cells are grown in Dulbecco's modification of minimum essential medium (MEM) with 10% fetal bovine serum (FBS), 0.1 mM nonessential amino acids, 10 U/ml penicillin and 10 micrograms/ml streptomycin. All cells are grown and maintained as described by V. Seagroatt and D. I. Magrath, J. Biol. Standardization 11:47-54 (1983); and P. Y. Shi et al., J. Virology, 76:5847-5856 (2002), the disclosure of which is hereby incorporated by reference in its entirety.

The effect of various antiviral therapies on the replication capacity of YFV is determined as follows. The vaccine strain (17D) of Yellow Fever Virus is propagated on vero cells as described by V. Seagroatt and D. I. Magrath J. Biol. Standardization 11:47-54 (1983), the disclosure of which are hereby incorporated by reference in their entirety. Infectious virus is collected from cell supernatants, purified and titered.

Next, separate cultures of vero cells are maintained in growth medium supplemented with the concentrations of antiviral compounds indicated in Table 2 above. The cells are pretreated prior to infection in growth medium supplemented with antiviral compounds and under 5% CO₂ and at 37 degrees C. After three days subconfluent cells are infected at a multiplicity of infection (MOI) of 5 in triplicate wells with the Yellow Fever Virus stock. Next, the infected cells are maintained in growth medium supplemented with the antiviral compounds and incubated under 5% CO₂ and at 37 degrees C. for up to 124 hours. Aliquots of medium are removed at 12-24 hour intervals and stored at −70 degrees C. Harvested virus is used to perform plaque assays on PS cells to compare the replication capacity of the viruses grown on the various treatments shown in Table 2 as described. See A. T. De Madrid and J. S. Porterfield, Bull. W.H.O. 40:113-121 (1969); V. Seagroatt and D. I. Magrath, J. Biol. Standardization 11:47-54 (1983), the disclosures of which are hereby incorporated by reference in their entirety.

We claim:

1. A method for treating an RNA virus infection in a mammal comprising administering a therapeutically effective amount of a combination of a ribonucleoside analog in association with an inhibitor of IMPDH.

2. The method of claim 1, wherein the ribonucleoside analog is ribavirin or a derivative thereof or a pharmaceutically acceptable salt thereof.

3. The method of claim 1, wherein the inhibitor of IMPDH is mycophenolic acid or a derivative thereof.

4. The method of claim 1, wherein the inhibitor of IMPDH is (S)-N-3-[3-(3-methoxy-4-oxazol-5-yl-phenyl)ureido]-benzylcarbamic acid tetrahydrofuran-3-yl-ester.

5. The method of claim 1, wherein the combination further comprises an interferon.

6. The method of claim 5, wherein said interferon is interferon-alpha.

7. The method of claim 6, wherein said interferon-alpha is pegylated.

8. The method of claim 1, wherein the viral infection is caused by a virus selected from the group consisting of Hepatitis C Virus, West Nile Virus, Dengue Virus, Yellow Fever Virus, Bovine Viral Diarrhea Virus and Venezuelan Equine Encephalitis Virus.

9. A method for treating a viral infection in a mammal comprising administering to a mammal in need of such treatment a therapeutically effective amount of a combination of ribavirin in association with (S)-N-3-[3-(3-methoxy-4-oxazol-5-yl-phenyl)ureido]-benzylcarbamic acid tetrahydrofuran-3-yl-ester and pegylated interferon-alpha-2b.

10. A method for decreasing side effects associated with ribavirin antiviral therapy in a mammal comprising administering from about 60 mg/day to about 600 mg/day of ribavirin in association with an inhibitor of IMPDH.

11. The method of claim 10, wherein said inhibitor of IMPDH is (S)-N-3-[3-(3-methoxy-4-oxazol-5-yl-phenyl)ureido]-benzylcarbamic acid tetrahydrofuran-3-yl-ester.

12. The method of claim 10, further comprising administering a therapeutically effective amount of an interferon.

13. The method of claim 12, wherein said interferon is interferon-alpha.

14. The method of claim 13, wherein said interferon-alpha is pegylated.

15. The method of claim 10, wherein said anti-viral therapy is directed to an infection with by a virus selected from the group consisting of Hepatitis C Virus, West Nile Virus, Dengue Virus, Yellow Fever Virus, Bovine Viral Diarrhea Virus and Venezuelan Equine Encephalitis Virus.

16. A method for decreasing side effects of a ribavirin antiviral therapy in a mammal comprising administering a dose of from about 60 mg/day to about 600 mg/day of ribavirin in association with (S)-N-3-[3-(3-methoxy-4-oxazol-5-yl-phenyl)ureido]-benzylcarbamic acid tetrahydrofuran-3-yl-ester and pegylated interferon-alpha-2b.

17. A composition comprising:
(a) from about 60 mg to about 600 mg of ribavirin; and
(b) a therapeutically effective amount of (S)-N-3-[3-(3-methoxy-4-oxazol-5-yl-phenyl)ureido]-benzylcarbamic acid tetrahydrofuran-3-yl-ester.

18. The composition of claim 17, wherein said composition further comprises a pharmaceutically acceptable carrier.

19. The composition of claim 17, further comprising a pharmaceutically acceptable carrier and from about 1 mg to about 6000 mg of said (S)-N-3-[3-(3-methoxy-4-oxazol-5-yl-phenyl)ureido]-benzylcarbamic acid tetrahydrofuran-3-yl-ester.

20. The composition of claim 17, wherein the amount of (S)-N-3-[3-(3-methoxy-4-oxazol-5-yl-phenyl)ureido]-benzylcarbamic acid tetrahydrofuran-3-yl-ester is in the range of from about 150 mg to about 5000 mg.


SEQUENCE LISTING (3 sequences)

SEQ ID NO: 1 - DNA, 17 bp, Hepatitis C virus
atggacaggcgccctga

SEQ ID NO: 2 - DNA, 20 bp, Hepatitis C virus
ttgatgggcagcttggtttc

SEQ ID NO: 3 - DNA, 17 bp, Hepatitis C virus
cacgccatgcgctgcgg